(19) United States
(12) Patent Application Publication
(10) Pub. No.: US 2024/0316041 A1
(43) Pub. Date: Sep. 26, 2024

(54) PROCESS FOR THE PREPARATION OF SITAGLIPTIN FREE FROM GENOTOXIC IMPURITIES

(71) Applicant: Zydus Lifesciences Limited, Ahmedabad (IN)

(72) Inventors: Kumar Kamlesh SINGH, Ahmedabad (IN); Santosh Devidas DIWAKAR, Ahmedabad (IN); Chintan Sureshbhai DHOLAKIA, Ahmedabad (IN); Abhishek Kumar LAL, Ahmedabad (IN); Dayawant Bhimsing RATHOD, Ahmedabad (IN); Hitesh Phulchandbhai THUMBAR, Ahmedabad (IN)

(21) Appl. No.: 18/594,459

(22) Filed: Mar. 4, 2024

(30) Foreign Application Priority Data
Mar. 3, 2023 (IN) 202321014441

(51) Int. Cl. A61K 31/4985; C07D 487/04

(52) U.S. Cl. CPC A61K 31/4985; C07D 487/04

(57) ABSTRACT

The present invention relates to sitagliptin or a pharmaceutically acceptable salt thereof substantially free from genotoxic impurities particularly nitrosamine impurities and processes for its preparation. In particular, the invention relates to process for the preparation of high purity sitagliptin free from nitrosamine impurities and other genotoxic and carcinogenic impurities below threshold concentration.

### FIELD OF THE INVENTION

The present invention relates to sitagliptin or a pharmaceutically acceptable salt thereof substantially free from genotoxic impurities particularly nitrosamine impurities and processes for its preparation. In particular, the invention relates to a process for the preparation of high purity sitagliptin free from nitrosamine impurities and other genotoxic and carcinogenic impurities below threshold concentration.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Sitagliptin chemically known as 7-[(3R)-3-amino-1-oxo-4-(2,4,5-trifluorophenyl) butyl]-5,6,7,8-tetrahydro-3-(trifluoromethyl)-1,2,4-triazolo[4,3-a]pyrazine and structurally represented as the compound of Formula I below:

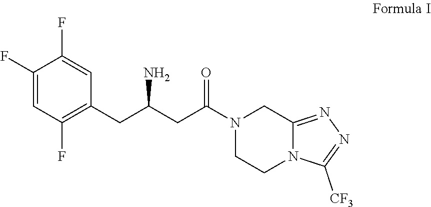 [SMILES: N[C@@H](CC(=O)N1CCn2c(nnc2C(F)(F)F)C1)Cc1cc(F)c(F)cc1F]

is an oral antihyperglycemic (antidiabetic) drug of the dipeptidyl peptidase-4 (DPP-4) inhibitor class. It was approved by USFDA and marketed under the trade name of JANUVIA®. The JANUVIA® tablets contain sitagliptin in the form of sitagliptin phosphate monohydrate. This enzyme-inhibiting drug is used either alone or in combination with other oral antihyperglycemic agents (such as metformin or a thiazolidinedione) for treatment of diabetes mellitus type 2.

Sitagliptin phosphate monohydrate has the following structural Formula (Ia).

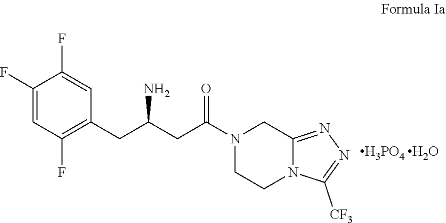

U.S. Pat. No. 6,699,871 B2 (the U.S. '871 patent) discloses sitagliptin and process for its preparation.

U.S. Pat. No. 7,326,708 B2 (the U.S. '708 patent) discloses dihydrogenphosphate salt of sitagliptin and crystalline hydrates thereof, in particular a crystalline monohydrate.

Nitrosamine impurities are defined as probably carcinogenic compounds to humans. Several nitrosamine impurities are identified by various regulatory agencies. USFDA has identified seven nitrosamine impurities that theoretically could be present in drug products: N-Nitrosodimethylamine (NDMA), N-nitrosodiethylamine (NDEA), N-nitroso-N-methyl-4-aminobutanoic acid (NMBA), N-nitrosoisopropylethyl amine (NIPEA), N-nitrosodiisopropylamine (NDIPA), N-nitrosodibutylamine (NDBA), and N-nitrosomethylphenylamine (NMPA). Five of them (NDMA, NDEA, NMBA, NIPEA, and NMPA) have actually been detected in drug substances or drug products.

Various regulatory agencies have recommended that there should be appropriate control strategies to prevent or restrict the presence of nitrosamine impurities as much as possible and, where necessary, improve their manufacturing processes. Companies should also evaluate the risk of nitrosamines being present in their medicines and carry out appropriate tests.

USFDA recently became aware of a nitrosamine impurity, Nitroso-STG-19 (known as NTTP), in certain samples of sitagliptin. To avoid a shortage and to ensure patients have access to an adequate supply of the medicine, FDA has decided not to object to the temporary distribution of sitagliptin containing NTTP above the acceptable intake limit of 37 ng per day, and up to 246.7 ng per day. The nitrosamine impurity, Nitroso-STG-19 can be structurally represented as a compound of Formula III as shown below:

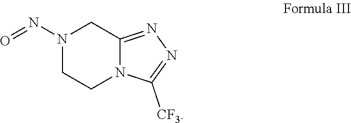 [SMILES: O=NN1CCn2c(nnc2C(F)(F)F)C1]

The present inventors have also determined the content of NTTP in the marketed product of sitagliptin tablets i.e. Januvia®. The results are as shown below:

| | Lot | | | NTTP content |
|---|---|---|---|---|
| Product | Number | Expiry Date | Pack | (ppm) |
| Januvia ® | U032037 | March 2024 | 90's | 0.89 |
| Tablets 25 | W008455 | October 2024 | Count | 0.59 |
| mg | U024078 | March 2024 | | 0.92 |
| Januvia ® | U037629 | July 2024 | | 0.98 |
| Tablets 50 | W015409 | September 2024 | | 0.51 |
| mg | W015410 | September 2024 | | 0.61 |
| Januvia ® | U036568 | July 2024 | | 1.82 |
| Tablets 100 | U039898 | September 2024 | | 0.67 |
| mg | W008877 | October 2024 | | 1.69 |

As shown in above table, the marketed product of sitagliptin was found to have NTTP content much above the acceptable limit of 0.37 ppm.

Therefore, there is need to develop a process that could remove the nitrosamine impurity of Formula III from the sitagliptin or a pharmaceutical acceptable salt thereof to an acceptable level.

### SUMMARY OF THE INVENTION

In one general aspect, the present invention provides sitagliptin or a pharmaceutically acceptable salt thereof substantially free of nitrosamine impurity of Formula III,

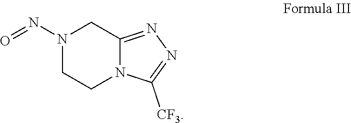 [SMILES: O=NN1CCn2c(nnc2C(F)(F)F)C1]

In another general aspect, the present invention provides sitagliptin or a pharmaceutically acceptable salt thereof substantially free of impurity of Formula II or nitrosamine impurity of Formula III,

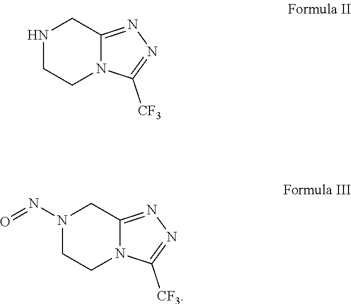 [SMILES: FC(F)(F)c1nnc2n1CCNC2 ; O=NN1CCn2c(nnc2C(F)(F)F)C1]

In another general aspect, the present invention provides sitagliptin or a pharmaceutically acceptable salt thereof having a purity of about 99.5% or more and a chiral purity of about 99.5% or more as determined by area percentage of high-performance liquid chromatography (HPLC), and containing about 0.37 ppm or less of nitrosamine impurity of Formula III,

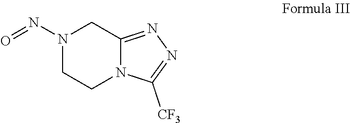 [SMILES: O=NN1CCn2c(nnc2C(F)(F)F)C1]

as determined by a LCMS method.

In another general aspect, the present invention provides sitagliptin or a pharmaceutically acceptable salt thereof having a purity of about 99.5% or more and a chiral purity of about 99.5% or more and about 100 ppm or less of the impurity of Formula II or a salt thereof,

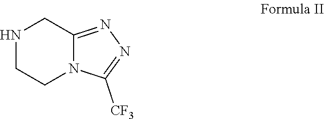 [SMILES: FC(F)(F)c1nnc2n1CCNC2]

as determined by area percentage of high-performance liquid chromatography (HPLC).

In another general aspect, the present invention provides a process for the preparation of sitagliptin or a pharmaceutically acceptable salt thereof substantially free of nitrosamine impurity of Formula III,

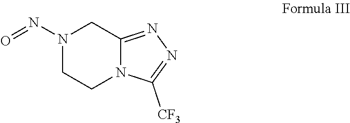 [SMILES: O=NN1CCn2c(nnc2C(F)(F)F)C1]

the process comprising:
(a) reacting sitagliptin with phosphoric acid to obtain phosphoric acid salt of sitagliptin;
(b) treating the phosphoric acid salt of sitagliptin with a suitable base to obtain sitagliptin containing about 0.37 ppm or less of nitrosamine impurity of Formula III; and
(c) optionally, converting sitagliptin obtained at step b, to its pharmaceutically acceptable salts.

In another general aspect, the present invention provides sitagliptin or a pharmaceutically acceptable salt thereof having a purity of about 99.5% or more and a chiral purity of about 99.5% or more as determined by area percentage of high-performance liquid chromatography (HPLC), and containing about 0.37 ppm or less of nitrosamine impurity of Formula III,

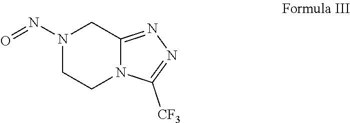 [SMILES: O=NN1CCn2c(nnc2C(F)(F)F)C1]

as determined by a LCMS method, wherein the sitagliptin or a pharmaceutically acceptable salt thereof is prepared by a process comprising:
(a) reacting sitagliptin with phosphoric acid to obtain phosphoric acid salt of sitagliptin;
(b) treating the phosphoric acid salt of sitagliptin with an aqueous base at a pH of about 9 to 14 to obtain a reaction mixture;
(c) treating the reaction mixture with one or more organic solvents and separating the aqueous and organic layers;
(d) recovering the sitagliptin having a purity of about 99.5% or more and a chiral purity of about 99.5% or more, and containing about 0.37 ppm or less of nitrosamine impurity of Formula III, by the removal of the organic solvents; and
(e) optionally, converting the sitagliptin obtained at step (d) to its pharmaceutically acceptable salt.

In another general aspect, the present invention provides the use of compound of Formula II or salt thereof,

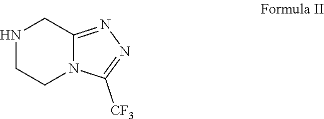 [SMILES: FC(F)(F)c1nnc2n1CCNC2]

for the preparation of sitagliptin or a pharmaceutically acceptable salt thereof, wherein the compound of Formula II is substantially free of nitrosamine impurity of Formula III,

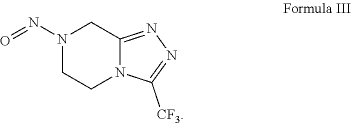 [SMILES: O=NN1CCn2c(nnc2C(F)(F)F)C1]

In another general aspect, the present invention provides a pharmaceutical composition comprising sitagliptin or a pharmaceutically acceptable salt thereof substantially free of nitrosamine impurity of Formula III,

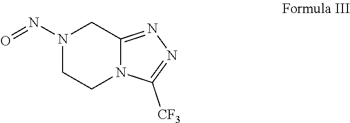 [SMILES: O=NN1CCn2c(nnc2C(F)(F)F)C1]

and one or more pharmaceutically acceptable excipients.

In another general aspect, the present invention provides a pharmaceutical composition comprising sitagliptin or a pharmaceutically acceptable salt thereof having a purity of about 99.5% or more and a chiral purity of about 99.5% or more as determined by area percentage of high-performance liquid chromatography (HPLC), and one or more pharmaceutically acceptable excipients, wherein the pharmaceutical composition contains about 0.37 ppm or less of nitrosamine impurity of Formula III,

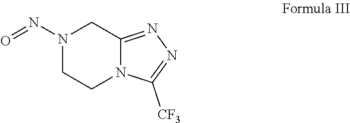 [SMILES: O=NN1CCn2c(nnc2C(F)(F)F)C1]

wherein the nitrosamine impurity of Formula III is determined by a LCMS method.

In another general aspect, the present invention provides a method of treating diabetes mellitus type 2 comprising administering to a patient in need thereof a pharmaceutical composition comprising a therapeutically effective amount of sitagliptin or a pharmaceutically acceptable salt thereof substantially free of nitrosamine impurity of Formula III and one or more pharmaceutically acceptable excipients.

In another general aspect, the present invention provides a method for treating diabetes mellitus type 2 comprising administering to a patient in need thereof a pharmaceutical composition comprising sitagliptin or a pharmaceutically acceptable salt thereof having a purity of about 99.5% or more and a chiral purity of about 99.5% or more as determined by area percentage of high-performance liquid chromatography (HPLC), and one or more pharmaceutically acceptable excipients, wherein the pharmaceutical composition contains about 0.37 ppm or less of nitrosamine impurity of Formula III,

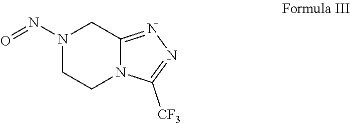 [SMILES: O=NN1CCn2c(nnc2C(F)(F)F)C1]

wherein the nitrosamine impurity of Formula III is determined by a LCMS method.

### DETAILED DESCRIPTION OF THE INVENTION

The term “substantially free of nitrosamine impurity of Formula III,” as used herein refers to said compound containing the nitrosamine impurity of Formula III in an amount less than the threshold of toxicological concern limit (TTC) i.e. 0.37 ppm or less by area percentage of LCMS. In particular, less than about 0.2 ppm, less than about 0.1 ppm, particularly not in detectable amount by area percentage of LCMS or absent.

The term “aqueous base” as used herein refers to an aqueous solution of one or more bases as described herein in the specification. Alternatively, aqueous base can also refers to an aqueous solution of one or more bases formed in-situ in the reaction mixture by adding water soluble one or more bases to the reaction mixture having water as one of the solvent.

The terms “treating”, or “reacting” as used herein have meanings as widely used by general prior art in the field of invention and can be easily understood by those skilled in the art.

The term, “obtain”, or “obtaining” as used herein may include filtration, filtration under vacuum, centrifugation, evaporation, distillation, distillation under vacuum, and/or decantation for isolation of the product. The product obtained may be further or additionally dried to achieve the desired moisture values. For example, the product may be dried in a tray drier, dried under vacuum and/or in a Fluid Bed Drier. The product may be preceded for further reaction with or without isolation and with or without drying in case of the product was isolated.

The term “about” as used herein is intended to mean approximately, in the region of, roughly, or around. When the term “about” is used in conjunction with a numerical range, it modifies that range by extending the boundaries above and below the numerical values set forth. In general, the term “about” may be used herein to modify a numerical value above and below the stated value by a variance of, for example, 10%.

The term “pharmaceutically acceptable” as used herein indicates that the material does not have properties that would cause one of skill in the art to avoid administration of the material to a patient, taking into consideration the disease or conditions to be treated and the respective route of administration. Further, the material is considered to be safe for administration in humans or animals.

The term “method of treatment” as used herein, means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms.

The pharmaceutically acceptable salt of sitagliptin includes the acid additional salt of sitagliptin such as sitagliptin phosphate or sitagliptin hydrochloride, etc.

In general, sitagliptin used as starting material in the process of the present invention may be prepared by following the process as disclosed in U.S. Pat. No. 7,326,708 B2.

### Abbreviations

HPLC: High Performance Liquid Chromatography
LCMS: Liquid Chromatography Mass Spectrometry
ppm: Parts Per Million
TTC=Threshold of Toxicological Concern
NDMA=N-Nitrosodimethylamine
NDEA=N-Nitrosodiethylamine
NMBA=N-nitroso-N-methyl-4-aminobutanoic acid
ND=not (non) detectable

In general, the solvents may be removed from the reaction mixture in order to obtain solid or precipitate. The solvents may be removed by one or more of filtration, filtration under vacuum, centrifugation, decantation, distillation and distillation under vacuum.

The product(s) obtained may further be converted to any other physical forms thereof which includes but not specifically limited to salt(s), solvate(s), hydrate(s), co-crystal(s) and solid dispersion(s) in either crystalline or amorphous forms.

Thus, in one general aspect, the present invention provides sitagliptin or a pharmaceutically acceptable salt thereof substantially free of nitrosamine impurity of Formula III,

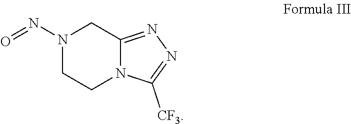 [SMILES: O=NN1CCn2c(nnc2C(F)(F)F)C1]

In another general aspect, the present invention provides sitagliptin or a pharmaceutically acceptable salt thereof containing about 0.37 ppm or less of nitrosamine impurity of Formula III,

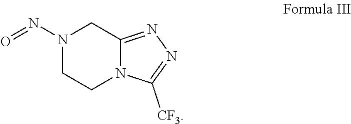 [SMILES: O=NN1CCn2c(nnc2C(F)(F)F)C1]

In one embodiment, the present invention provides sitagliptin or a pharmaceutically acceptable salt thereof containing less than about 0.37 ppm of nitrosamine impurity of Formula III, for example, less than about 0.3 ppm, less than about 0.25 ppm, less than about 0.2 ppm, less than about 0.15 ppm, less than about 0.1 ppm, less than about 0.05 ppm, or absent as determined by LCMS.

In another embodiment, sitagliptin or a pharmaceutically acceptable salt thereof the present invention is having a purity of about 99% or more as determined by area percentage of HPLC, for example, a purity of about 99.5% or more, about 99.8% or more, or about 100% as determined by area percentage of HPLC.

In another embodiment, sitagliptin or a pharmaceutically acceptable salt thereof the present invention is having a chiral purity of about 99.5% or more as determined by area percentage of HPLC, for example a chiral purity of about 99.7%, about 99.85% or about 100% as determined by area percentage of HPLC.

In another embodiment, sitagliptin or a pharmaceutically acceptable salt thereof of the present invention is having a purity of about 99.5% or more and a chiral purity of about 99.5% or more as determined by area percentage of HPLC.

In another embodiment, sitagliptin or a pharmaceutically acceptable salt thereof of the present invention is having a purity of about 99.85% or more and a chiral purity of about 99.85% or more as determined by area percentage of HPLC.

In another embodiment, sitagliptin or a pharmaceutically acceptable salt thereof of the present invention contains less than about 500 ppm of the impurity of Formula II or a salt thereof, for example, less than about 400 ppm, less than about 300 ppm, less than about 200 ppm, less than about 100 ppm, less than about 50 ppm, less than about 10 ppm, less than about 5 ppm or not in detectable amount by area percentage of HPLC.

Particularly, sitagliptin or pharmaceutically acceptable salt thereof of the present invention contains less than about 100 ppm of the impurity of Formula II or a salt thereof as determined by area percentage of HPLC. More particularly, sitagliptin or pharmaceutically acceptable salt thereof of the present invention contains less than about 50 ppm of the impurity of Formula II or a salt thereof as determined by area percentage of HPLC.

In another embodiment, sitagliptin or pharmaceutically acceptable salt thereof of the present invention contains less than about 10 ppm of the impurity of Formula II or a salt thereof, more particularly less than about 5 ppm or not in detectable amount of the impurity of Formula II or a salt thereof, as determined by area percentage of HPLC.

In another embodiment, the present invention provides sitagliptin or a pharmaceutically acceptable salt thereof having a purity of about 99.5% or more and a chiral purity of about 99.5% or more and about 100 ppm or less of the impurity of Formula II or a salt thereof,

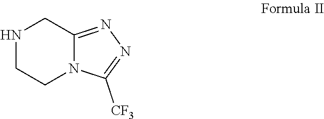 [SMILES: FC(F)(F)c1nnc2n1CCNC2]

as determined by area percentage of high-performance liquid chromatography (HPLC).

In another general aspect, the present invention provides a process for the preparation of sitagliptin or a pharmaceutically acceptable salt thereof substantially free of nitrosamine impurity of Formula III,

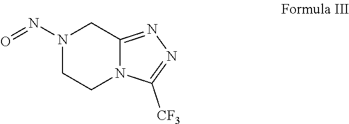 [SMILES: O=NN1CCn2c(nnc2C(F)(F)F)C1]

the process comprising:
(a) reacting sitagliptin with phosphoric acid to obtain phosphoric acid salt of sitagliptin;
(b) treating the phosphoric acid salt of sitagliptin with a suitable base to obtain sitagliptin containing about 0.37 ppm or less of nitrosamine impurity of Formula III; and
(c) optionally, converting sitagliptin obtained at step b, to its pharmaceutically acceptable salt.

In one embodiment, the obtained sitagliptin or a pharmaceutically acceptable salt thereof contains about 0.37 ppm or less of nitrosamine impurity of Formula III.

In another embodiment, the obtained sitagliptin or a pharmaceutically acceptable salt thereof contains less than about 0.37 ppm of nitrosamine impurity of Formula III, for example, less than about 0.3 ppm, less than about 0.25 ppm, less than about 0.2 ppm, less than about 0.15 ppm, less than about 0.1 ppm, less than about 0.05 ppm, or absent as determined by LCMS.

In another embodiment, the step (a) is performed in-situ. In another embodiment, phosphoric acid salt of sitagliptin at step (a) is not isolated.

In general, step (a) is carried out in one or more solvents selected from water, methanol, ethanol, 2-propanol, or mixtures thereof. Particularly, the solvent is a mixture of water and 2-propanol.

In one embodiment, step (a) of the above process is carried out in one or more solvents wherein solvent is a mixture of 2-propanol and water used in a ratio of about 5:1 to about 9:1 v/v respectively. More particularly, the 2-propanol and water are used in a ratio of 7:1 v/v respectively.

In general step (a) of the above process is carried out by:
(i) heating a mixture of sitagliptin with phosphoric acid in one or more solvents at a temperature of about 50° C. to about 80° C.;
(ii) stirring the reaction mixture at about 50° C. to about 80° C. for about 15 minutes to 1 hour; and
(iii) slowly cooling the reaction mixture to a temperature of about 0° C. to about 10° C. to obtain phosphoric acid salt of sitagliptin.

In general, the base at step (b) is selected from one or more of hydroxides, carbonates of alkali metals and ammonia. Particularly, one or more bases is selected from one or more of sodium hydroxide, potassium hydroxide, ammonium hydroxide, sodium carbonate, potassium carbonate, sodium bicarbonate, potassium bicarbonate, ammonia gas, ammonia solution or mixtures thereof. More particularly, the base is sodium hydroxide.

In general step (b) is carried out by treating the phosphoric acid salt of sitagliptin with one or more bases in a mixture of water and one or more organic solvents to obtain a pH of about 9 to about 14, particularly to a pH of about 12 to 13.5. In particular, the one or more organic solvents for the purpose can be selected from dichloromethane, ethyl acetate, chloroform, dichloroethane, diethylether, methyl tert-butyl ether, and toluene. After reaching to the desired pH, the reaction mixture can be further stirred and then organic layer can be separated and concentrated under reduced pressure to obtain sitagliptin substantially free of nitrosamine impurity of Formula III.

In general, sitagliptin substantially free of nitrosamine impurity of Formula III can be converted to its pharmaceutically acceptable salt such as sitagliptin phosphate by following the process as disclosed in U.S. Pat. No. 9,409,912 B2.

In another general aspect, the present invention provides a process for the preparation of sitagliptin or a pharmaceutically acceptable salt thereof having a purity of about 99.5% or more and a chiral purity of about 99.5% or more as determined by area percentage of high-performance liquid chromatography (HPLC), and containing about 0.37 ppm or less of nitrosamine impurity of Formula III,

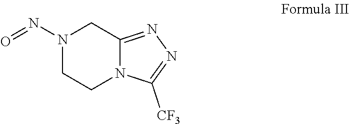 [SMILES: O=NN1CCn2c(nnc2C(F)(F)F)C1]

as determined by a LCMS method, wherein the sitagliptin or a pharmaceutically acceptable salt thereof is prepared by a process comprising:
(a) reacting sitagliptin with phosphoric acid to obtain phosphoric acid salt of sitagliptin;
(b) treating the phosphoric acid salt of sitagliptin with an aqueous base at a pH of about 9 to 14 to obtain a reaction mixture;
(c) treating the reaction mixture with one or more organic solvents and separating the aqueous and organic layers;
(d) recovering the sitagliptin having a purity of about 99.5% or more and a chiral purity of about 99.5% or more, and containing about 0.37 ppm or less of nitrosamine impurity of Formula III, by the removal of the organic solvents; and
(e) optionally, converting the sitagliptin obtained at step (d) to its pharmaceutically acceptable salt.

In another embodiment, the obtained sitagliptin or a pharmaceutically acceptable salt thereof contains less than about 0.37 ppm of nitrosamine impurity of Formula III, for example, less than about 0.3 ppm, less than about 0.25 ppm, less than about 0.2 ppm, less than about 0.15 ppm, less than about 0.1 ppm, less than about 0.05 ppm, or absent as determined by LCMS.

In another embodiment, the step (a) is performed in-situ. In another embodiment, phosphoric acid salt of sitagliptin at step (a) is not isolated.

In general, step (a) is carried out in one or more solvents selected from water, methanol, ethanol, 2-propanol, or mixtures thereof. Particularly, the solvent is a mixture of water and 2-propanol.

In one embodiment, step (a) of the above process is carried out in one or more solvents wherein solvent is a mixture of 2-propanol and water used in a ratio of about 5:1 to about 9:1 v/v respectively. More particularly, the 2-propanol and water are used in a ratio of about 7:1 v/v respectively.

In general step (a) of the above process is carried out by a process comprising:
(i) heating a mixture of sitagliptin with phosphoric acid in one or more solvents at a temperature of about 50° C. to about 80° C.;
(ii) stirring the reaction mixture at about 50° C. to about 80° C. for about 15 minutes to 1 hour; and
(iii) slowly cooling the reaction mixture to a temperature of about 0° C. to about 10° C.

In general, the aqueous base at step (b) is selected from one or more of hydroxides, carbonates of alkali metals and ammonia.

In an embodiment, hydroxides are selected from one or more of sodium hydroxide, potassium hydroxide, or ammonium hydroxide, carbonates are selected from one or more of sodium carbonate, potassium carbonate, sodium bicarbonate, or potassium bicarbonate, ammonia is ammonia gas, or ammonia solution, or mixtures thereof. More particularly, the aqueous base is sodium hydroxide.

In general, the step (b) of above process is carried out by treating the phosphoric acid salt of sitagliptin with an aqueous base so as to adjust the pH of resultant reaction mixture to about 9 to 14, particularly to a pH of about 12 to 13.5.

In general, the reaction mixture obtained at step (b) is treated with one or more organic solvents. The one or more organic solvents for the purpose can be selected from dichloromethane, ethyl acetate, chloroform, dichloroethane, diethylether, methyl tert-butyl ether, and toluene. Particularly, the organic solvent is dichloromethane. The resultant reaction mixture can be further stirred and then aqueous and organic layer can be separated as per step (c) of the above process.

In general, the step (b) of the above process is carried out by treating the phosphoric acid salt of sitagliptin with an aqueous base so as to adjust the pH of resultant reaction mixture to about 9 to 14 and then it was treated with one or more organic solvents as per step (c) of the above process. Alternatively, the phosphoric acid salt of sitagliptin can be treated with one or bases in a mixture of water and one or more organic solvents so as to adjust the pH of about 9 to 14 and then the aqueous and organic layers can be separated.

Particularly, the step (b) and (c) is collectively carried out by treating the phosphoric acid salt of sitagliptin with one or more bases in a mixture of water and one or more organic solvents to obtain a pH of about 9 to 14 and separating the aqueous and organic layers.

In general, the removal of organic solvents at step (d) of the above process is carried out by one or more of distillation, distillation under vacuum, evaporation, spray drying, agitated thin film drying, freeze drying, filtration, filtration under vacuum, centrifugation, or decantation. In particular, by distillation or distillation under vacuum.

In general, sitagliptin obtained at step (d) can be converted to its pharmaceutically acceptable salt such as sitagliptin phosphate by following the process as disclosed in U.S. Pat. No. 9,409,912 B2.

In another general aspect, the present invention provides use of compound of Formula II or salt thereof,

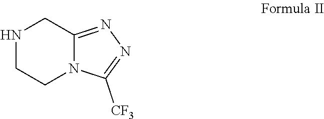 [SMILES: FC(F)(F)c1nnc2n1CCNC2]

for the preparation of sitagliptin or a pharmaceutically acceptable salt thereof, wherein the compound of Formula II or salt thereof is substantially free of nitrosamine impurity of Formula III,

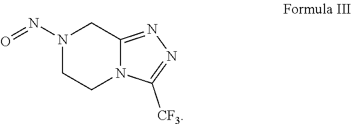 [SMILES: O=NN1CCn2c(nnc2C(F)(F)F)C1]

In another general aspect, the present invention provides use of compound of Formula II or salt thereof,

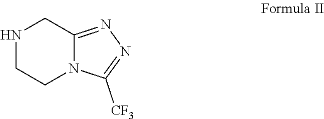 [SMILES: FC(F)(F)c1nnc2n1CCNC2]

for the preparation of sitagliptin or a pharmaceutically acceptable salt thereof, wherein the compound of Formula II or salt thereof is containing about 0.37 ppm or less of nitrosamine impurity of Formula III.

In another embodiment, the compound of Formula II or salt thereof contains less than about 0.37 ppm of nitrosamine impurity of Formula III, for example, less than about 0.3 ppm, less than about 0.25 ppm, less than about 0.2 ppm, less than about 0.15 ppm, less than about 0.1 ppm, less than about 0.05 ppm, or absent as determined by LCMS.

The compound of Formula II can be converted to sitagliptin or a pharmaceutically acceptable salt thereof by following the process as disclosed in U.S. Pat. No. 9,409,912 B2.

In another general aspect, the present invention provides a pharmaceutical composition comprising sitagliptin or a pharmaceutically acceptable salt thereof, and one or more pharmaceutically acceptable excipients, wherein the pharmaceutical composition contains about 0.37 ppm or less of nitrosamine impurity of Formula III,

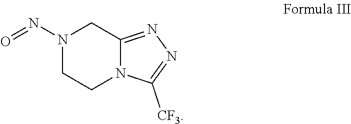 [SMILES: O=NN1CCn2c(nnc2C(F)(F)F)C1]

In another general aspect, the present invention provides a pharmaceutical composition comprising sitagliptin having a purity of about 99% or more by area percentage of high-performance liquid chromatography (HPLC) or a pharmaceutically acceptable salt thereof and nitrosamine impurity of Formula III present in an amount of about 0.37 ppm or less.

In another general aspect, the present invention provides a pharmaceutical composition comprising sitagliptin or a pharmaceutically acceptable salt thereof having a purity of about 99.5% or more and a chiral purity of about 99.5% or more by area percentage of HPLC and nitrosamine impurity of Formula III present in an amount of about 0.37 ppm or less.

In another embodiment, the present invention provides a pharmaceutical composition comprising sitagliptin or a pharmaceutically acceptable salt thereof containing less than about 0.37 ppm of nitrosamine impurity of Formula III, for example, less than about 0.3 ppm, less than about 0.25 ppm, less than about 0.2 ppm, less than about 0.15 ppm, less than about 0.1 ppm, less than about 0.05 ppm, or absent as determined by LCMS.

In another general aspect, the present invention provides a pharmaceutical composition comprising sitagliptin or a pharmaceutically acceptable salt thereof having a purity of about 99.5% or more and a chiral purity of about 99.5% or more as determined by area percentage of high-performance liquid chromatography (HPLC), and one or more pharmaceutically acceptable excipients, wherein the pharmaceutical composition contains about 0.37 ppm or less of nitrosamine impurity of Formula III,

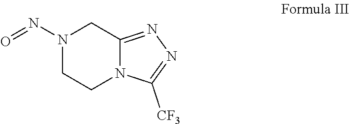 [SMILES: O=NN1CCn2c(nnc2C(F)(F)F)C1]

wherein the nitrosamine impurity of Formula III is determined by a LCMS method.

The one more pharmaceutically acceptable excipients that are useful for preparing the pharmaceutical composition include, but are not limited to one or more diluents, binders, disintegrants, glidants, lubricants, sweeteners/taste masking agents, compression aids, colorants, flavors and the like. Suitable diluents include, but are not limited to, saccharides, including monosaccharides, disaccharides, polysaccharides and sugar alcohols such as arabinose, lactose, dextrose, sucrose, fructose, maltose, mannitol, erythritol, sorbitol, xylitol, lactitol, and other bulking agents such as powdered cellulose, microcrystalline cellulose, starch, dibasic calcium phosphate, tribasic calcium phosphate, calcium carbonate, dextrose, kaolin, magnesium carbonate, magnesium oxide, purified sugar and derivatives thereof. Suitable binders, which include, but are not limited to, methyl cellulose, hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinylpyrrolidone, starch, pregelatinized starch, polyvinyl alcohol, tragacanth, sodium alginate, gums, synthetic resins, silicic acid, hydrophilic polymers and the like. Suitable disintegrants which include, but are not limited to, croscarmellose sodium, crospovidone, sodium starch glycolate, corn starch, potato starch, maize starch and modified starches like pregelatinized starch, calcium silicates, low-substituted hydroxypropylcellulose and the like. Suitable lubricants and glidants which include, but are not limited to, talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like. Suitable taste masking agents may include one or more of polymers, sweeteners and flavors.

The pharmaceutical composition of sitagliptin or a pharmaceutical acceptable salt thereof may be developed in the form of tablets, capsules, powders, pellets, granules, microspheres, minitablets or any suitable solid unit forms known to person skilled in the art; mouth dissolving tablets; dispersible tablets; effervescent tablets; trilayer tablets; inlay tablets. In particular, the pharmaceutical composition is in the form of tablet or capsule.

The pharmaceutical composition of sitagliptin or a pharmaceutically acceptable salt thereof may be manufactured by using various techniques known to the person skilled in the art, such as, but not limited to direct compression, wet granulation, dry granulation, hot melt granulation, hot melt extrusion, fluidized bed granulation, extrusion, and solvent evaporation.

In another general aspect, the present invention provides a method for treating diabetes mellitus type 2 comprising administering to a patient in need thereof a pharmaceutical composition comprising a therapeutically effective amount of sitagliptin or a pharmaceutically acceptable salt thereof, and one or more pharmaceutically acceptable excipients; wherein the pharmaceutical composition contains about 0.37 ppm or less of nitrosamine impurity of Formula III,

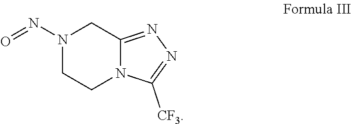 [SMILES: O=NN1CCn2c(nnc2C(F)(F)F)C1]

The nitrosamine impurity of Formula III in the sitagliptin or a pharmaceutically acceptable salt thereof or in the pharmaceutical composition thereof was determined by LCMS method using ESI mode with mobile phase.

In general, the LCMS method for the determination of compound of Formula III comprises: HPLC system equipped with a triple quadrupole MS/MS detector with ESI mode, MRM scan, C18 or modified C18 column, mobile phase A being aqueous acidic buffer solution and mobile phase B being methanol or acetonitrile, and using gradient elution.

In general, aqueous acidic buffer of mobile phase A of the LCMS method is selected from aqueous formic acid, acetic acid, etc.

In one embodiment, the aqueous acidic buffer of mobile phase A of the LCMS method is aqueous formic acid, and the mobile phase B is methanol.

In general, the C18 column used in LCMS method is having a length of about 150 mm, a diameter of about 4.6 mm and a particle size of 3 u.

In particular, the triple quadrupole MS/MS detector in the LCMS method is Q-trap 4500 (AB Sciex).

In another embodiment, the triple quadrupole MS/MS detector in the LCMS method is Q-trap 4500 (AB Sciex) and C18 column is having a length of about 150 mm, a diameter of about 4.6 mm and a particle size of 3 u.

### The Analytical Method for the Determination of Nitrosamine Impurity of Formula III Chromatographic Conditions:

Equipment: HPLC system equipped with MS detector.
Column: ACE-3-PFP C18 (150 mm×4.6 mm, 3.0 um)
Wavelength: NA
Flow Rate: 0.8 mL/min
Column temp.: 40° C.
Sampler cooler: 10° C.
Injection Volume: 20 HL
Run time: 20 Minutes
Washing vial: Diluent
Gradient Programme:

| Time | Mobile phase A | Mobile phase B |
|---|---|---|
| 0.01 | 85 | 15 |
| 10 | 50 | 50 |
| 15 | 10 | 90 |
| 15.5 | 85 | 15 |
| 20 | 85 | 15 |

### Diluent Preparation:

A mixture of 500 mL of water and 500 mL of methanol was prepared.

### Mass Parameter:

Equipment: Q-trap 4500 (AB Sciex)
Scan type: MRM
Source: ESI

### Preparation of Blank:

Diluent was used as blank.

### Preparation of Mobile Phase-A

1.0 mL of formic acid was transferred into 1000 mL of water and mixed.

### Preparation of Mobile Phase-B

Methanol was used as such.

### HPLC Method for the Determination of Purity and Content of Compound of Compound of Formula II in the Sitagliptin or a Pharmaceutically Acceptable Salt Thereof:

Column: Zorbax SB-C8 (250 mm×4.6 mm, 5 μm), wavelength:210 nm
Buffer: Accurately 1 mL of triethylamine was transferred into a suitable container containing about 1000 mL of Milli-Q water and mixed well. About 1 g of 1-Octane sulphonic acid sodium salt was added into the same container and sonicated to dissolve the content and mixed well. The pH was adjusted to 2.5+0.05 with control addition of dilute orthophosphoric acid solution and mixed well and then filtered through 0.45 μm membrane filter paper.
Mobile phase A: buffer solution as a mobile phase-A.
Mobile phase B: acetonitrile as a mobile phase-B. Gradient Elution.

### HPLC Method for the Determination of Chiral Purity of Sitagliptin

Column: Chiralpak ADH (250 mm×4.6 mm, 5 m), wavelength:268 nm
Mobile Phase: Mixture of diethylamine:deionized water:n-heptane:ethanol in the ratio of 1:1:400:600.

The present invention is further illustrated by the following example which is provided merely to be exemplary of the invention and do not limit the scope of the invention. Certain modification and equivalents will be apparent to those skilled in the art and are intended to be included within the scope of the present invention. The examples are set forth to aid in understanding the invention but are not intended to, and should not be construed to limit its scope in any way. The examples do not include detailed descriptions of conventional methods. Such methods are well known to those of ordinary skill in the art and are described in various publications.

### EXAMPLES

### Example 1: Purification of Sitagliptin

Isopropyl alcohol (175 mL) and purified water (25 mL) were charged into the reactor. Sitagliptin free base (25 g) was charged into the reactor at temperature 30° C.±5° C. The reaction mass was stirred for 15 minutes at temperature 30° C.±5° C. 85% Phosphoric acid (8.4 g, 1.4 equivalent) was charged into the reactor within 15 to 30 min at a temperature 30° C.±5° C. The reaction mass was stirred for 15 minutes at temperature 30° C.±5° C. The reaction mass was heated up to temperature 70° C.±5° C. The reaction mass was stirred for 30 minutes at temperature 70° C.±5° C. The reaction mass was cooled up to temperature 5° C.±5° C. The reaction mass was stirred for 30 minutes at temperature 5°+5° C. The reaction mass was centrifuged, the wet caked was washed with chilled isopropyl alcohol. The Purified water (125 mL) was charged into the reactor. The wet cake was charged into the reactor. Dichloromethane (50 mL) was charged into the reactor. Then 40% sodium hydroxide solution (35 mL) was added into the reactor at temperature 30° C.±5° C. and pH of reaction mass was adjusted between 12 and 13.5. The reaction mass was stirred for 15 minutes at temperature 30° C.±5° C. The lower organic layer was separated; the aqueous layer was kept into the reactor at temperature 30° C.±5° C. Dichloromethane was charged into the reactor containing aqueous layer at temperature 30° C.±5° C. The reaction mass was stirred for 15 minutes at temperature 30° C.±5° C. The reaction mass was settled for 15 minutes at temperature 30° C.±5° C. The organic layer was separated and upper aqueous layer was discarded. The combined organic layer was again washed with water.

Dichloromethane was distilled out below 55° C. till 70% of the volume has been distilled out atmospherically. Then dichloromethane was distilled out below 55° C. under vacuum till semisolid mass obtained.

Isopropyl alcohol (50 mL) was charged into the reactor at below 55° C. Isopropyl alcohol was distilled out under vacuum at below 55° C. to remove the traces of dichloromethane. Isopropyl alcohol (12.5 mL) and cyclohexane (125 mL) were charged into the reactor at below 55° C. The reaction mass was heated up to 71° C.±4° C. The reaction mass was stirred for 60 min at temperature 71° C.±4° C. The reaction mass was cooled up to 10° C.±5° C. The reaction mass was stirred for 60 min, at temperature 10° C.±5° C. The reaction mass was centrifuged and the wet cake was washed with chilled cyclohexane. The wet cake was spin dried and further dried under vacuum into the vacuum tray dryer for 4 hrs to obtain sitagliptin free base.
Content of nitrosamine impurity of Formula III: 0.005 ppm.
Content of impurity of Formula II: 5 ppm.
HPLC purity: 100%.
Chiral Purity: 100%.

### Example 2: Preparation of Sitagliptin Phosphate Monohydrate

The sitagliptin free base obtained in the above example is converted to sitagliptin phosphate monohydrate by following the analogues process disclosed in U.S. Pat. No. 9,409,912 B2.
Content of nitrosamine impurity of Formula III: 0.007 ppm.
Content of impurity of Formula II: 2.18 ppm.
HPLC purity: 99.97%.
Chiral Purity: 100%.

While the present invention has been described in terms of its specific embodiments, certain modifications and equivalents will be apparent to those skilled in the art and are intended to be included within the scope of the present invention.

1. Sitagliptin or a pharmaceutically acceptable salt thereof having a purity of about 99.5% or more and a chiral purity of about 99.5% or more as determined by area percentage of high-performance liquid chromatography (HPLC), and containing about 0.37 ppm or less of nitrosamine impurity of Formula III, 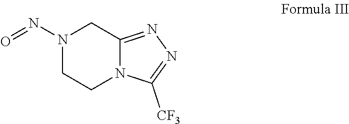 [SMILES: O=NN1CCn2c(nnc2C(F)(F)F)C1]
as determined by a LCMS method.

2. The sitagliptin or a pharmaceutically acceptable salt thereof according to claim 1, having a purity of about 99.85% or more and a chiral purity of about 99.85% or more as determined by area percentage of HPLC.

3. Sitagliptin or a pharmaceutically acceptable salt thereof having a purity of about 99.5% or more and a chiral purity of about 99.5% or more and about 100 ppm or less of the impurity of Formula II or a salt thereof, 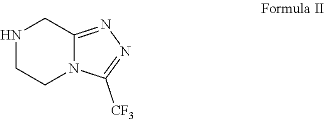 [SMILES: FC(F)(F)c1nnc2n1CCNC2]
as determined by area percentage of high-performance liquid chromatography (HPLC).

4. The sitagliptin or a pharmaceutically acceptable salt thereof according to claim 1, wherein the LCMS method for the determination of compound of Formula III comprises: HPLC system equipped with a triple quadrupole MS/MS detector with ESI mode, MRM scan, C18 or modified C18 column, mobile phase A being aqueous acidic buffer solution, and mobile phase B being methanol or acetonitrile, and using gradient elution.

5. The sitagliptin or a pharmaceutically acceptable salt thereof according to claim 4, wherein the aqueous acidic buffer of mobile phase A is aqueous formic acid, and the mobile phase B is methanol.

6. The sitagliptin or a pharmaceutically acceptable salt thereof according to claim 4, wherein the triple quadrupole MS/MS detector is Q-trap 4500 (AB Sciex) and C18 column is having a length of about 150 mm, a diameter of about 4.6 mm and a particle size of 3μ.

7. Sitagliptin or a pharmaceutically acceptable salt thereof having a purity of about 99.5% or more and a chiral purity of about 99.5% or more as determined by area percentage of high-performance liquid chromatography (HPLC), and containing about 0.37 ppm or less of nitrosamine impurity of Formula III, 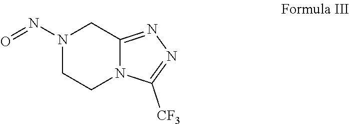 [SMILES: O=NN1CCn2c(nnc2C(F)(F)F)C1]
as determined by a LCMS method, wherein the sitagliptin or a pharmaceutically acceptable salt thereof is prepared by a process comprising:
(a) reacting sitagliptin with phosphoric acid to obtain phosphoric acid salt of sitagliptin;
(b) treating the phosphoric acid salt of sitagliptin with an aqueous base at a pH of about 9 to 14 to obtain a reaction mixture;
(c) treating the reaction mixture with one or more organic solvents and separating the aqueous and organic layers;
(d) recovering the sitagliptin having a purity of about 99.5% or more and a chiral purity of about 99.5% or more, and containing about 0.37 ppm or less of nitrosamine impurity of Formula III, by the removal of the organic solvents; and
(e) optionally, converting the sitagliptin obtained at step (d) to its pharmaceutically acceptable salt.

8. The sitagliptin or a pharmaceutically acceptable salt thereof according to claim 7, wherein the step (a) is performed in-situ.

9. The sitagliptin or a pharmaceutically acceptable salt thereof according to claim 7, wherein the step (a) is carried out in one or more solvents selected from water, methanol, ethanol, 2-propanol, or mixtures thereof.

10. The sitagliptin or a pharmaceutically acceptable salt thereof according to claim 7, wherein the aqueous base at step (b) is selected from one or more of hydroxides, carbonates of alkali metals, and ammonia.

11. The sitagliptin or a pharmaceutically acceptable salt thereof according to claim 10, wherein the hydroxides are selected from one or more of sodium hydroxide, potassium hydroxide, or ammonium hydroxide, carbonates are selected from one or more of sodium carbonate, potassium carbonate, sodium bicarbonate, or potassium bicarbonate, ammonia is ammonia gas, or ammonia solution, or mixtures thereof.

12. The sitagliptin or a pharmaceutically acceptable salt thereof according to claim 9, wherein the solvent is a mixture of 2-propanol and water used in a ratio of about 5:1 to about 9:1 v/v, respectively.

13. The sitagliptin or a pharmaceutically acceptable salt thereof according to claim 7, wherein the step (a) is carried out by a process comprising:
i. heating a mixture of sitagliptin with phosphoric acid in one or more solvents at a temperature of about 50° C. to about 80° C.;
ii. stirring the reaction mixture at about 50° C. to about 80° C. for about 15 minutes to 1 hour; and
iii. slowly cooling the reaction mixture to a temperature of about 0° C. to about 10° C.

14. The sitagliptin or a pharmaceutically acceptable salt thereof according to claim 7, wherein the step (b) and (c) is collectively carried out by treating the phosphoric acid salt of sitagliptin with one or more bases in a mixture of water and one or more organic solvents to obtain a pH of about 9 to 14 and separating the aqueous and organic layers.

15. The sitagliptin or a pharmaceutically acceptable salt thereof according to claim 7, wherein the removal of organic solvents at step (d) is carried out by one or more of distillation, distillation under vacuum, evaporation, spray drying, agitated thin film drying, freeze drying, filtration, filtration under vacuum, centrifugation, or decantation.

16. A pharmaceutical composition comprising sitagliptin or a pharmaceutically acceptable salt thereof having a purity of about 99.5% or more and a chiral purity of about 99.5% or more as determined by area percentage of high-performance liquid chromatography (HPLC), and one or more pharmaceutically acceptable excipients, wherein the pharmaceutical composition contains about 0.37 ppm or less of nitrosamine impurity of Formula III, 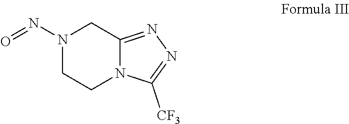 [SMILES: O=NN1CCn2c(nnc2C(F)(F)F)C1]
wherein the nitrosamine impurity of Formula III is determined by a LCMS method.

17. The pharmaceutical composition according to claim 16, wherein the pharmaceutically acceptable salt of sitagliptin is sitagliptin phosphate or sitagliptin hydrochloride.

18. A method for treating diabetes mellitus type 2 comprising administering to a patient in need thereof a pharmaceutical composition according to claim 16.
